(19) United States
(12) Patent Application Publication
(10) Pub. No.: US 2024/0350729 A1
(43) Pub. Date: Oct. 24, 2024

(54) PUMP DEVICE

(71) Applicant: AG IP HOLDING LLC, OKEMOS, MI (US)

(72) Inventor: Patrick Kenneth Powell, Grosse Pointe Park, MI (US)

(21) Appl. No.: 18/684,383

(22) PCT Filed: Aug. 17, 2022
(86) PCT No.: PCT/US22/40565

§ 371 Date: Feb. 16, 2024

Related U.S. Application Data
(60) Provisional application No. 63/233,929, filed on Aug. 17, 2021.
(60) Provisional application No. 63/233,930, filed on Aug. 17, 2021.
(60) Provisional application No. 63/233,932, filed on Aug. 17, 2021.
(60) Provisional application No. 63/233,934, filed on Aug. 17, 2021.
(60) Provisional application No. 63/257,648, filed on Oct. 20, 2021.
(60) Provisional application No. 63/270,783, filed on Oct. 22, 2021.
(60) Provisional application No. 63/286,182, filed on Dec. 6, 2021.

(51) Int. Cl. A61M 5/19; A61M 5/24; A61M 5/32; A61N 1/04

(52) U.S. Cl. CPC A61M 5/19; A61M 5/2425; A61M 5/3287; A61N 1/0452

(57) ABSTRACT

A device includes a pump body that has at least one variable-size compartment. The pump body is configured to be actuated to change the size of the at least one variable-size compartment. Flexible bladders are disposed in the at least one compartment. At least one common port is fluidly connected with interiors of the flexible bladders. The change in the size of the at least one variable-size compartment either compresses or expands the flexible bladders in unison and thereby, respectively, either expels the interiors through the at least one common port or draws a vacuum into the interiors through the at least one common port.

### BACKGROUND

A syringe is a pump that includes a plunger or piston that fits tightly within a cylindrical tube. The plunger is moveable inside of the tube to take in or expel liquid or gas through an orifice at one end of the tube. A needle, nozzle, or tubing may be fitted to the orifice to direct the flow into or out of the tube.

Syringes are ubiquitous in medicine for administering injections and intravenous therapy, and are used in other fields as well to apply compounds or draw and measure liquids, for example. Despite widespread use, syringes have drawbacks, such as but not limited to, being limited to cylindrical geometry, high forces to pump, easy to accidentally break, misalignment and variance in performance, large in size, pressure or suction that is limited by the cross-sectional area of the plunger, and difficult operation due to the long plunger stroke required.

### SUMMARY

A device according to an example of the present disclosure includes a pump body having at least one variable-size compartment. The pump body is configured to be actuated to change the size of the at least one variable-size compartment. Flexible bladders are disposed in the at least one compartment, and there is at least one common port fluidly connected with interiors of the flexible bladders. A change in the size of the at least one variable-size compartment either compresses or expands the flexible bladders in unison and thereby, respectively, either expels the interiors through the at least one common port or draws a vacuum into the interiors through the at least one common port.

In a further embodiment of any of the foregoing embodiments, the at least one variable-size compartment includes a plurality of variable-size compartments, the pump body includes a first rack having first arms and a second rack having second arms that are interleaved with the first arms so as to provide the variable-size compartments between the first arms and the second arms, and the flexible bladders are disposed, respectively, in the variable-size compartments.

In a further embodiment of any of the foregoing embodiments, actuation of the pump body moves the first arms toward the second arms and thereby reduces the sizes of the variable-size compartments to compress the flexible bladders.

A further embodiment of any of the foregoing embodiments includes at least one needle connected with the at least one common port and defining a needle axis. The pump body defines a centerline axis. The movement of the first rack relative to the second rack is in a direction parallel to the centerline axis, and the needle axis is non-parallel to the centerline axis.

In a further embodiment of any of the foregoing embodiments, the pump body defines a centerline axis, the movement of the first rack relative to the second rack is in a direction parallel to the centerline axis, and the at least one port is offset from the centerline axis.

In a further embodiment of any of the foregoing embodiments, for an amount of the movement over a distance (d) and a number N of the flexible bladders, there is a change in the total volume of the interiors of the flexible bladders (ΔVₜₒₜₐₗ), and a ratio of ΔVₜₒₜₐₗ/d is N:1.

A further embodiment of any of the foregoing embodiments includes at least one needle connected with the at least one common port.

In a further embodiment of any of the foregoing embodiments, actuation of the pump body to reduce the size of the at least one variable-size compartment compresses the flexible bladders, and the flexible bladders contain a liquid medicament that is expelled through the common port.

In a further embodiment of any of the foregoing embodiments, at least one of the flexible bladders contains a powder.

In a further embodiment of any of the foregoing embodiments, the interiors of the flexible bladders are fluidly connected in series with each other.

In a further embodiment of any of the foregoing embodiments, the flexible bladders are connected in series by convolutions, and further include stents in the convolutions preventing collapse of the convolutions upon actuation of the pump body.

In a further embodiment of any of the foregoing embodiments, the interiors of the flexible bladders are fluidly connected in parallel with the common port.

A further embodiment of any of the foregoing embodiments includes an actuator operable to actuate the pump body.

In a further embodiment of any of the foregoing embodiments, the actuator includes a spring.

In a further embodiment of any of the foregoing embodiments, the actuator is in the at least one variable-size compartment.

In a further embodiment of any of the foregoing embodiments, at least two of the flexible bladders are of unequal volume.

In a further embodiment of any of the foregoing embodiments, the flexible bladders are formed of a multi-layer bladder wall.

A further embodiment of any of the foregoing embodiments includes an inflatable needle fluidly connected with the at least one port.

In a further embodiment of any of the foregoing embodiments, one of the flexible bladders contains a first medicament and the another one of the flexible bladders contains a second, different medicament.

A further embodiment of any of the foregoing embodiments includes a needle fluidly connected with the at least one port and a pad that is adhesively attachable to tissue. The pad has first and second electrodes that are spaced apart from each other, an electric power source connected with the first and second electrodes, and a through hole for receiving the needle there through into the tissue.

A further embodiment of any of the foregoing embodiments includes a needle connected with the at least one port. The needle includes an electrical circuit that is open by default, and an electric power source connected with the needle, wherein insertion of the needle into a location of a subject animal to inject a medicament through the needle closes the circuit and produces an electric field in a vicinity of the location that dilates blood vessels and stimulates muscle contractions to aid in delivery of the medicament to the subject animal.

A further embodiment of any of the foregoing embodiments includes a plenum fluidly interconnecting the flexible bladders to the at least one common port.

A device according to an example of the present disclosure includes an actuator and a pump including a pump body that has a first rack having first arms and a second rack having second arms that are interleaved with the first arms such that there are compartments between the first arms and the second arms. The first rack and the second rack are moveable relative to each other such that compartments are variable in size. Flexible bladders are disposed in the compartments. The flexible bladders contain at least one liquid, a plenum fluidly interconnects interiors of the flexible bladders, and at least one common port is fluidly connected with the interiors of the flexible bladders via the plenum. The actuator is operable to actuate the pump body and thereby cause the first arms to move closer to the second arms to reduce the size of the compartments and thus compress the flexible bladders to expel the liquid from the interiors through the plenum to the at least one common port.

In a further embodiment of any of the foregoing embodiments, the actuator comprises a bias member that has stored potential energy.

In a further embodiment of any of the foregoing embodiments, the bias member includes a spring.

In a further embodiment of any of the foregoing embodiments, the flexible bladders include in the interiors a shape recovery member configured to expand the flexible bladders after compression.

A method for using a device according to an example of the present disclosure includes providing a device as in any of the foregoing embodiments and actuating the pump body to compress or expand the flexible bladders in unison and thereby, respectively, either expel the interiors through the at least one common port or draw a vacuum into the interiors through the at least one common port.

In a further example of any of the above or below embodiments, the movement is rotary.

In a further example of any of the above or below embodiments, the pump body is disposed in a dental frame configured to fit on a dental arch of a user.

A further example of any of the above or below embodiments further comprises a needle connected with the at least one port, an electrically conductive sheath around at least a portion of the needle, a spring disposed around the needle, and an electric power source connected with the needle via the spring.

A further example of any of the above or below embodiments further comprises a fluid pump including a first tube, a second tube inside of the first tube, a reservoir defined between the first tube and the second tube, an inlet port into the second tube, and an outlet port from the reservoir, wherein movement of the first rack relative to the second rack causes the flexible bladders to pressurize the second tube to thereby expel the reservoir through the outlet port.

In a further example of any of the above or below embodiments, the second tube is convoluted.

In a further example of any of the above or below embodiments, on opposite sides of the first arms from the compartments there are additional compartments between the first arms and the second arms, further comprising balloons disposed in the additional compartments, and a compressed gas source in communication with the balloons.

A further example of any of the above or below embodiments further comprises a spacer between the first rack and the second rack, the spacer having a height that determines the size of the compartments.

In a further example of any of the above or below embodiments, the flexible bladders include at least one inlet port, and further comprising a vial of medicament that is mateable with the at least one inlet port to fill the interiors with liquid.

In a further example of any of the above or below embodiments, the pump body defines a centerline axis, the movement of the first rack relative to the second rack is in a direction parallel to the centerline axis, and the flexible bladders are asymmetric about the centerline axis.

In a further example of any of the above or below embodiments, the pump body includes a one-way inter-lock that locks the first rack and the second rack together upon movement past a lock position preventing reverse movement of the first rack relative to the second rack.

In a further example of any of the above or below embodiments, the flexible bladders include a phase change material that blocks fluid connection between the interiors of the flexible bladders and the at least one port, the phase change material being responsive to a temperature change to fluidly connect the interiors of the flexible bladders with the at least one port.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1A illustrates a pump device.

FIG. 1B illustrates a sectioned view of the pump device of FIG. 1A.

FIG. 1C illustrates the pump device of FIG. 1B without the flexible bladders.

FIG. 1D illustrates the flexible bladders of the pump device of FIG. 1B.

FIG. 1E illustrates an example in which the needle is non-coaxial with the central axis of the device.

FIG. 2A illustrates a biased pump that has a bias member for actuation of the device.

FIG. 2B illustrates the biased pump upon actuation.

FIG. 3 illustrates the device with multiple needles.

FIG. 4 illustrates the device with an array of micro-needles.

FIG. 5 illustrates the device with multiple bias members.

FIG. 6A illustrates another example of a flexible bladder that is rectangular.

FIG. 6B illustrates another example of a flexible bladder that is asymmetrical.

FIG. 7 illustrates a sectioned view of a bladder wall with a rebound bias.

FIG. 8A illustrates an expanded view of another example device that has flexible bladders configured in series.

FIG. 8B illustrates a fully assembled view of the device of FIG. 8A.

FIG. 9A illustrates an example of a syringe device that has flexible bladders configured in series.

FIG. 9B illustrates the device of FIG. 9A upon actuation.

FIG. 10A, 10B, and 10C illustrate an example device with a pad for generating an electric field.

FIG. 11A, 11B and 11c illustrate an inflatable needle.

In this disclosure, like reference numerals designate like elements where appropriate and reference numerals with the addition of one-hundred or multiples thereof designate modified elements that are understood to incorporate the same features and benefits of the corresponding elements.

### DETAILED DESCRIPTION

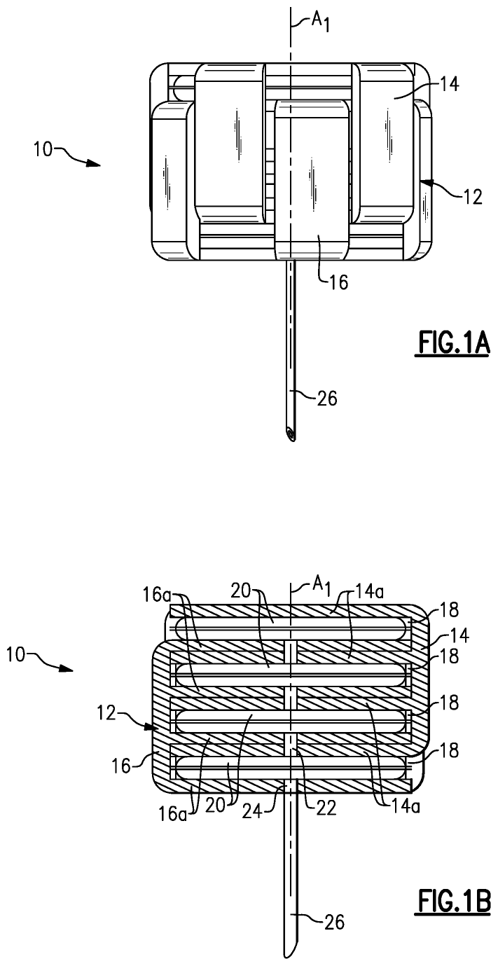

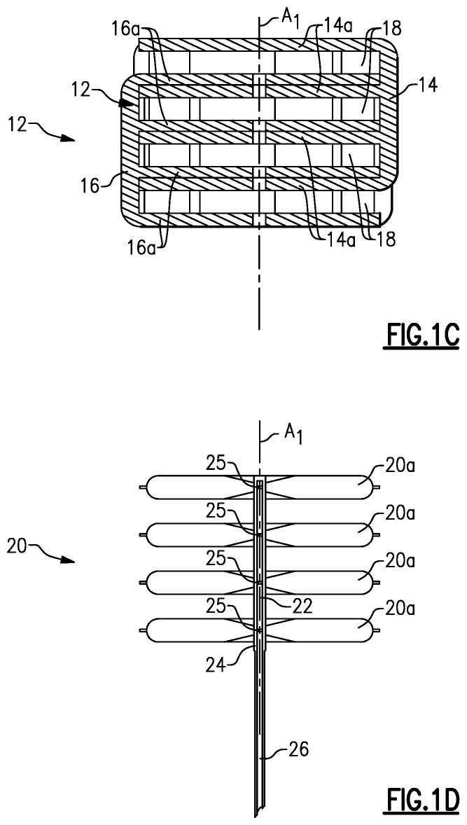

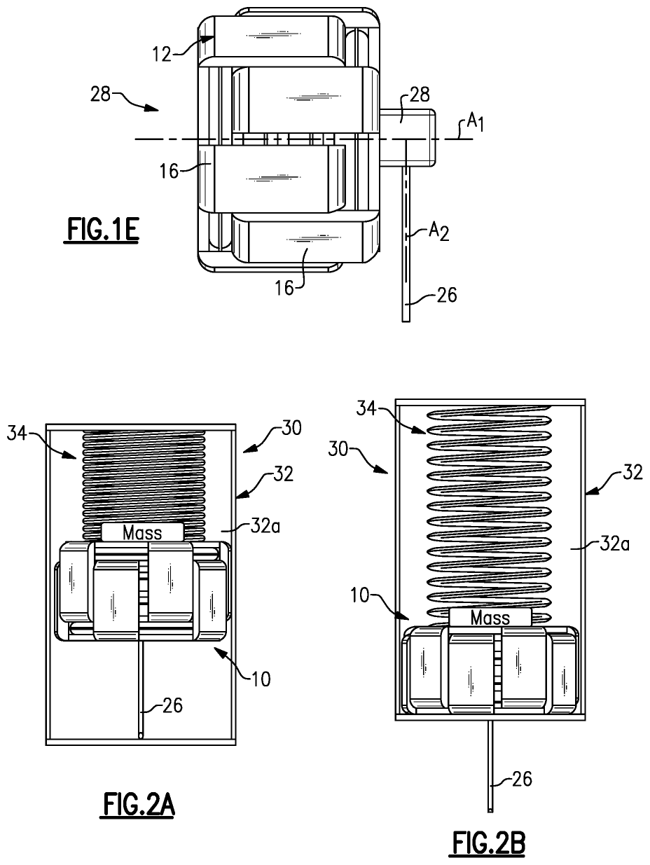

FIG. 1A illustrates an example of a device 10, and FIG. 1B illustrates a sectioned view of the device 10 taken along a plane containing the central axis A of the device 10. The device 10 includes a pump body 12 that is comprised of a first rack 14 and a second rack 16. The first rack 14 has a plurality of first arms 14a and the second rack 16 has a plurality of second arms 16b that are generally parallel to, and interleaved with, the first arms 14a so as to form compartments 18 there between. In this example, there are four such compartments 18, although it is to be understood that there could alternatively be anywhere from two to fifty (or more) compartments 18 depending on the numbers of arms 14a/16a provided on the racks 14/16.

In this example, the arms 14a/16a are of common geometry and the compartments 18 are thus also of common geometry from one to the next. As will be appreciated, however, the arms 14/16 could alternatively be of different geometries so that the compartments 18 are of different geometry from one to the next. In the illustrated example, the racks 14/16 (and thus also the pump body 12) are generally cylindrical about the central axis A such that the compartments 18 are cylindrical. The racks 14/16 could alternatively be rectangular, polygonal, or even irregularly-shaped as may be desired for adaption to a particular end use (e.g., a wearable).

The first rack 14 is moveable relative to the second rack 16 along the central axis A such that the sizes (volumes) of the compartments 18 are variable with movement between the racks 14/16. For instance, when the racks 14/16 are pushed together (e.g., from the top and bottom—FIG. 1B) the compartments 18 simultaneously reduce in size, and when the racks 14/16 are pulled apart the compartments 18 simultaneously increase in size.

The device 10 further includes flexible bladders 20 disposed in the compartments 18 (FIG. 1C illustrates the racks 14/16 without the flexible bladders 20, and FIG. 1D illustrates the flexible bladders 20 without the racks 14/16). Each flexible bladder 20 has an interior 20a, which may serve to contain a material, such as a liquid or combinations of a liquid and a powder. For instance, each compartment 18 contains at least one flexible bladder 20. The bladders 20 are flexible in that they are able to fully, or at least partially, conform to the sides of the compartments 18. For example, the flexible bladders 20 are formed of a polymeric material, such as but not limited to, ethylene vinyl alcohol or polyvinylidene fluoride.

In this example, the flexible bladders 20 are fluidly interconnected in parallel by a plenum 22 to at least one common port 24 (one shown). That is, flow to/from any one interior 20a to/from the plenum 22 need not go through any other interior 20a to get to/from the plenum 22. In general, the port 24 is or includes an orifice. For example, the orifice may be in one or both of the racks 14/16 or in a hub that is attached to the pump body 12. In the illustrated example, the port 24 is coaxial with the central axis A of movement of the pump body 12. It is to be understood, however, that the port 24 may alternatively be offset (non-coaxial) from the central axis A or that multiple ports 24 that are coaxial and/or non-coaxial may be used.

The pump body 12 is configured (e.g., by the interleaving and spacing of the arms 14a/16a of the racks 14/16) to be actuated to change the sizes of the compartments 18. As an example, actuation of the pump body 12 to push the racks 14/16 together causes the arms 14a/16a to move closer together, reducing the sizes of the compartments 18 and thereby compressing the flexible bladders 20 to simultaneously expel the interiors 20a through the port 24 via the plenum 22. For example, the liquid is expelled to a needle 26 for hypodermic injection. Alternatively, actuation of the pump body 12 to pull the racks 14/16 apart causes the arms 14a/16a to move apart, increasing the sizes of the compartments 18 and thereby expanding the flexible bladders 20 to draw a vacuum in the interiors 20a through port 24 via the plenum 22. In the latter example, the flexible bladders 20 will be attached to the arms 14a/16a, such as by adhesive.

The flexible bladders 20 may be used to deliver a liquid medicament, two or more different liquid medicaments that are unmixed with each other, or one or more liquid medicaments and one or more powders that are contained in the flexible bladders 20. For instance, one of the flexible bladders 20 may contain a first medicament and another of the flexible bladders 20 may contain a different medicament. The bladders 20 are initially sealed from each other such that the medicaments (or powder) do not intermix. However, upon actuation of the pump body 12, the seals break and the medicaments (or powder) mix and flow to the port 24. In this regard, the bladders 20 may all be of one common volume, or the bladders 20 may have different, unequal volumes. The device 10 may additionally or alternatively be used to produce a vacuum to draw a liquid or gas into the flexible bladders 20. In this case, the flexible bladders 20 are attached to the arms 14a/16a as mentioned above, so that movement of the arms 14a/16a farther apart expands the flexible bladders 20 to increase the volume of the interiors 20a and thus draw the vacuum.

Due to the plurality of flexible bladders 20, the required stroke is small relative to a typical syringe, while the resulting change in volume of the compartments is relatively large. For instance, for an amount of movement over a distance (d) of the first rack 14 relative to the second rack 16 there is a change in the total volume of the interiors 20a of the flexible bladders 20 (ΔVₜₒₜₐₗ). The ratio of movement is generally proportional to the number of bladders 20 employed, provided the bladders 20 are of equal volume. By example, a single bladder 20 would have a vol/d ratio of 1:1, whereas two bladders 20 would be a ratio of 2:1, three bladders would be 3:1, and so on and so forth.

The distance (d) of displacement is minimal and the device 10 can generate relatively high pressure or vacuum over short distances. Moreover, the change in total volume is generally proportional to the number of bladders 20, and is not proportional to the distance (d) as with known syringes. This advantageously enables higher pressure/vacuum and thereby may facilitate reducing needle diameter, which aids to reduce pain a patient experiences upon needle insertion. Additionally, the device 10 facilitates tolerance to misalignments between the racks 14/16, as the flexible bladders 20 are able to deform and thereby accommodate slight misalignments.

In the example of FIGS. 1A/1B, the port 24 and needle 26 are co-linear with each other and with the central axis A₁. However, in a modified example shown in FIG. 1E, there is a hub 28 that is attached to the pump body 12 and to which the needle 26 is attached. In this case, the needle 26 extends along needle axis A₂, which is approximately perpendicular to the central axis A of the pump body 12. Thus, the needle axis A₂ need not be co-axial with the central axis A along which the pump body 12 is actuated. As will be appreciated, the needle axis A₂ may be at any non-parallel angle to the axis A₁ or rotatable about the axis A₁ in order to adapt the device 10 for a particular implementation. In this regard, the device 10 can be readily designed for any of a variety of different possible configurations.

The device 10 may be incorporated into other structures or systems to facilitate and enhance functionality. For instance, as shown in FIGS. 2A and 2B the device 10 is incorporated into a biased pump 30. The biased pump 30 includes a housing 32 that has housings walls 32a that surround an interior region in which the device 10 and needle 26 are disposed. There is also an actuator 34 disposed in the interior region adjacent the device 10. In this example, the actuator 34 is a bias member and includes a spring. As used herein, an “actuator” refers to a mechanical device that supplies energy to mechanically operate the device 10. Example actuators may include, but are not limited to, coil springs, elastic devices or materials, compressed gas, electro-active polymers, electric motion, and servomechanisms.

Initially, in the example shown, the spring is in a compressed state between the device 10 and one of the walls 32a of the housing 32 so as to store potential energy, as is shown in FIG. 2A. Upon actuation of the spring to release the potential energy, as shown in FIG. 2B, the spring pushes the device 10 toward the other end of the housing 32, thereby causing the needle 26 to deploy from the housing 32 and penetrate into the subject. A manual or automated trigger mechanism may be used to release the spring. A pierceable seal may be provided at the point of deployment from the housing 32 to seal the interior region. The device 10 bottoms-out against the housing wall 32a, which pushes the racks 14/16 together as discussed above to expel the interiors 20a of the flexible bladders 20 through the needle 26.

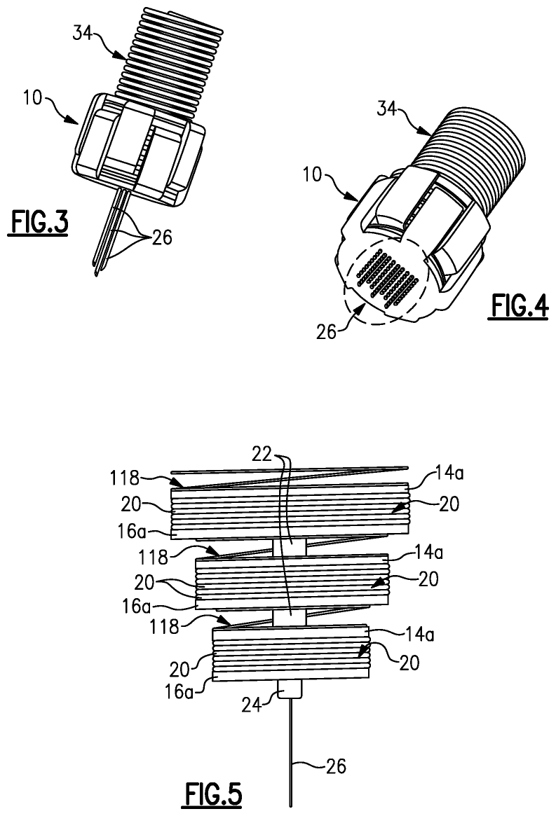

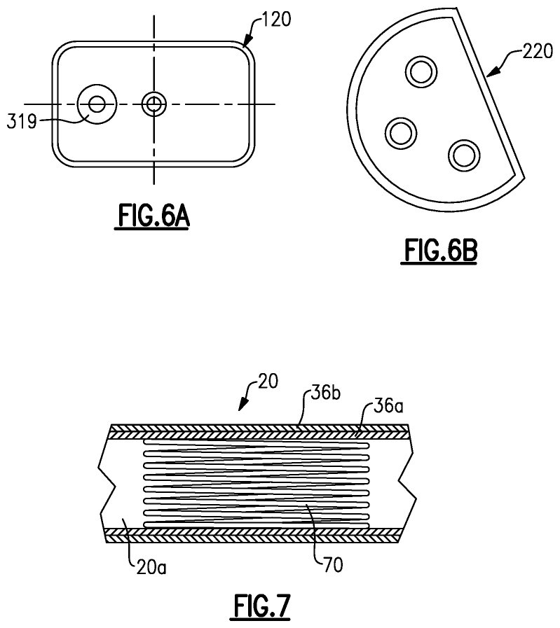

As can be appreciated, the initial position of the needle 26 within the housing 32 serves to protect the needle 26 from substances outside of the housing 32 and from undesired or accidental poking. In this regard, the biased pump 30 may be positioned at a location on the skin of a subject and then activated via the actuator 34 to cause the needle 26 to deploy and penetrate the subject to inject a medicament into the subject from the flexible bladders 20. In this regard, the device 10 may be adapted with a plurality of needles 26, as shown in FIG. 3, or with an array of micro-needles 26, as shown in FIG. 4. Additionally, with a reduced movement distance as mentioned above, the change in the spring force over the distance as compared to a longer distance is lessened, based on the spring factor k (F=k·d). Reduced variance in the force applied, and the resulting pressure developed, facilitates the precise dosing of a medicament.

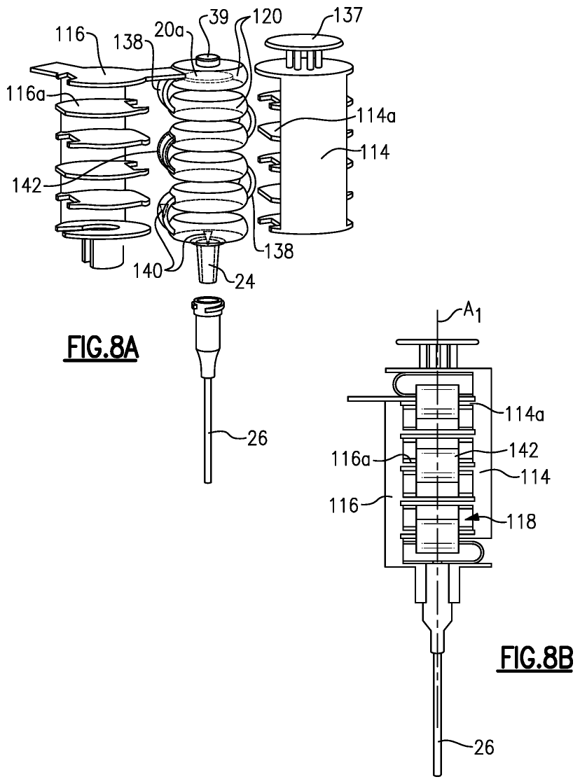

The device 10 can also be used with multiple actuators 34, such as shown in FIG. 5. In the illustrated view, the arms 14a/16a are shown but the remaining portions of the racks 14/16 are not shown. On opposite sides of the first arms 14a from the compartments 18 there are additional compartments 118 between the first arms 14a and the second arms 16a. There are multiple actuators 34 that are disposed in the compartments 118. In this example, the actuators 34 includes springs. Similar to the example of FIGS. 2A/2B, the springs are initially compressed to store potential energy such that upon activation the springs release the potential energy to move the arms 14a/16a closer together and compress the flexible bladders 20 (FIG. 5).

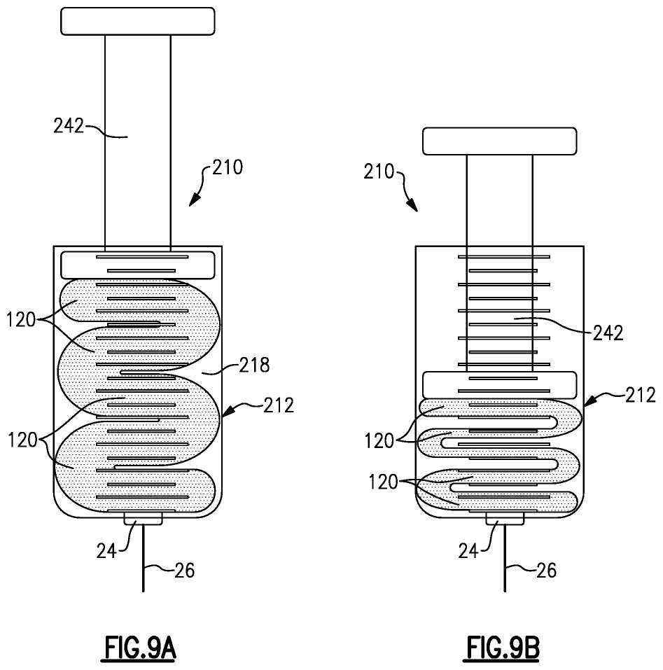

As indicated above, the racks 14/16 may be designed in cylindrical or other geometries for a particular end use. In similar regard, the geometry of the flexible bladders 20 may be adapted to the shape of the racks 14/16 and end use. For instance, FIG. 6A illustrates a representative flexible bladder 120 that is rectangular, and FIG. 6B illustrates a flexible bladder 220 that is asymmetric. The flexible bladders 20/120/220 have one or more orifices through which the plenum 22 extends and connects with the interior 20a. An additional orifice or orifices may be provided to accommodate other hardware in the device, release arms to trigger the actuator 34. An additional opening 319 may be provided to pass other device features through the bladder 20.

FIG. 7 illustrates a sectioned view through a representative portion of the wall of the flexible bladder 20. As shown, the wall may be of multi-layer construction and includes a first layer 36a that faces into the interior 20a of the bladder 20 and at least a second, outer layer 36b. For example, one or both of the layers 36a/36b is/are polymeric. In another example, one or both of the layers 36a/36b is/are metallic. In another example, one or both of the layers 36a/36b is/are inorganic, such as a thin, flexible glass. In another example, each layer 36a/36b is a different material selected from polymeric, metallic, and inorganic. In additional examples of any of the above examples, there are one or more additional layers that are either polymeric, metallic, or inorganic. One or more of the layers may be a polymeric, metallic, or inorganic material that has a low gas permeability in order to serve as a gas barrier, such as an oxygen barrier and/or low light permeability in order to serve as a light barrier, for example to block unwanted IR or UV irradiation. The multi-layer construction thus permits a composite approach to facilitate enhancement of one or more properties of the flexible bladders 20, such as strength, permeability, and elasticity. It is to be appreciated that although two layers are shown, additional layers may be used. In further examples, the bladder 20 contains a shape recovery bias 70, such as a spring, to assist with reinflating the bladder 20 after compression.

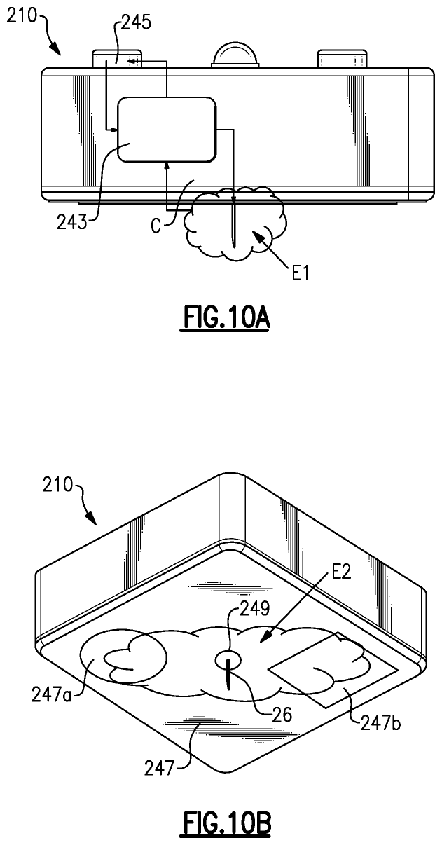

FIG. 8A illustrates an expanded view of another example device 110, and FIG. 8B illustrates the device 110 fully assembled. In this example, the racks 114/116 are axially longer in comparison to the racks 14/16 and thus have additional arms 114a/116a to form additional compartments 118. The first rack 114 also has a press tab 137 for manual actuation of the device 110. The flexible bladders 120 in this example are interconnected in series by convolutions 138 so as to form a serpentine configuration in which the bladders 20 are folded over onto one another. Additionally, there is no plenum as in the examples above. Rather, flow to/from any one interior 20a to/from the port 24 may go through another interior 20a to get to/from the port 24. For instance, in the illustrated example that has six bladders 120, a liquid that is in the first (top) bladder 120 will flow sequentially through the other five bladders 120 to reach the port 24. A liquid in the second bladder 120 (from the top) will flow sequentially through the other four bladders 120 to reach the port 24, and so on and so forth until the sixth bladder 120 (bottom).

Valves or burst seals 140 may be provided in the convolutions 138 or at other locations to prevent free flow of liquid between the bladders 120 before use, or to separate and prevent mixing of different liquids or powders that are contained in bladders 120. Additionally, a stent 142 may be disposed in each of the convolutions 138 to prevent collapse of the convolutions 138 upon actuation of the device 110, which might otherwise impede flow. An external fill port 39 may also be included to enable filling of the bladders 20.

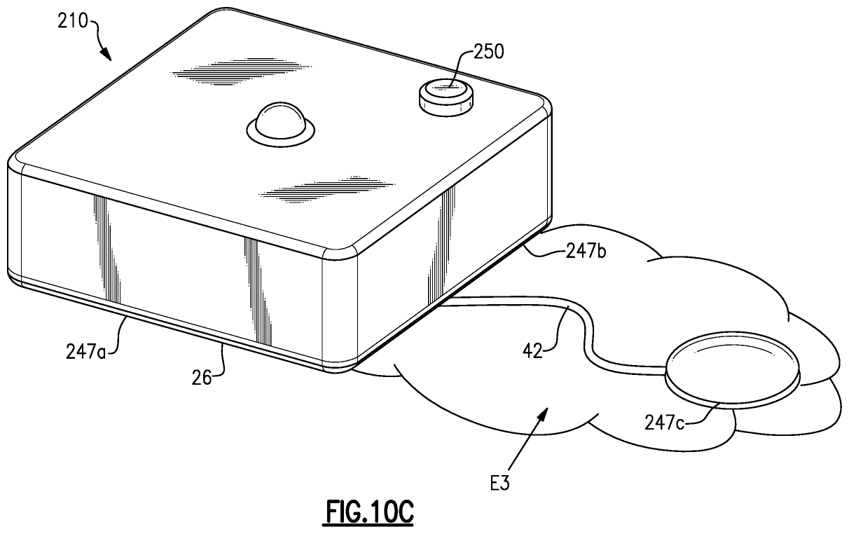

As shown in FIGS. 9A and 9B, the flexible bladders 120 that are configured in series may also be used in a syringe-type device 210. The device 210 includes a pump body 212 that defines a single interior compartment 218 in which the flexible bladders 120 are disposed. In this example, rather than the racks and arms, there is a plunger 242 that fits tightly in the pump body 212. Actuation of the plunger 242 over a stroke distance, as is shown in FIG. 9B, reduces the size of the compartment 218 and causes compression of the flexible bladders 120, thereby expelling the interiors 20a through the port 24. Although the syringe-style configuration may lengthen the stroke distance as compared to the prior examples, the design is somewhat simpler in that it does not require the racks and arms.

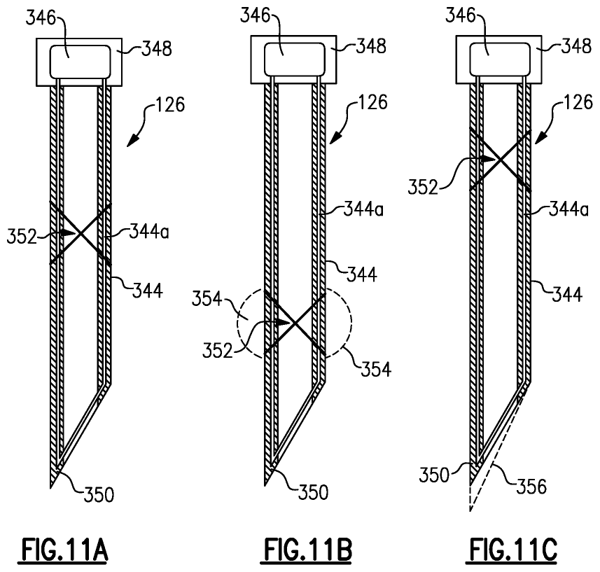

FIG. 10A illustrates another example of the device 210. In this example, the needle 26 is in an electric circuit C that is open by default. There is an electric power source 243 connected with the needle 26. Insertion of the needle 26 into a location of a subject animal (to inject a medicament through the needle 26) closes the circuit and produces an electric field E1 in a vicinity of the location. That is, the subject animal forms a bridge that closes the circuit. The electric field dilates blood vessels and contracts nearby muscles at the injection site to thereby aid in delivery of the medicament to the subject animal. The power source 243 may be connected with a trigger or switch 245 that the user can activate upon insertion of the needle 26. The device 210 is thus demonstrative of a method in which an electric field is applied at the injection site to facilitate drug delivery to the subject animal. Such a power source and electric field may also be used with any of the example devices herein.

FIG. 10B illustrates another example of the device 210. In this example, there is a pad 247 that is adhesively attachable to tissue. The pad 247 has first and second electrodes 247a/247b that are spaced apart from each other. The electric power source 243 is connected with the electrodes 247a/247b. There are one or more through-holes 249 in the pad 247 for receiving the needle 26 there through into the tissue. The hole or holes 249 may initially be sealed closed. Similar to the prior example, activation of the power source 243 generates an electric field E2 between the electrodes 247a/247b to dilate blood vessels, contract muscles, and aid drug delivery at the injection site. The approach is useful for wearable devices where the injection volume exceeds 1 ml or the injection time exceeds 30 seconds to increase physiological kinematic medicament distribution by increased blood flow to the injection site and faster disbursement into the greater body mass.

FIG. 10C illustrates another example of the device 210. In this example, there is a remote electrode pad 247c is adhesively attachable to tissue apart from the main device. The remote pad 247c may be electrically circuited with the device 210 including the needle 26, electrodes 247a/247b, and the battery 243 and may be remotely operated by a trigger. An electrical field E3 generated between the device 210 and remote electrode primarily in the skin sub-surfaces acts to dilate blood vessels and contract muscles to aid drug distribution from the injection site. The approach is useful for wearable devices where the injection volume exceeds 1 ml or the injection time exceeds 30 seconds to increase kinematic medicament absorption by increased blood flow to the injection site and faster disbursement into the greater body mass. The electrical stimulation may be continuous, periodic, pulse-width modulated, cyclical, or sinusoidal.

FIG. 11A illustrates an inflatable needle 126 that may be used with any of the devices herein or be used independently of the devices herein. The needle 126 includes a needle wall 344 that defines an internal channel 344a that is in fluid communication with one or more pressure sources 346 via hub 348. For instance, the needle wall 344 is composed of a polymeric material. The pressure source or sources 346 may be any of the devices disclosed herein, compressed gas source, or any mechanism that is capable of pumping fluid into the needle 126. The needle wall 344 is connected with a sharp needle tip 350. The needle tip 350 may be composed of a metallic material, such as stainless steel and may further include a low-friction treatment, such as a coating or lubricant.

Prior to inflation, the needle 126 is limp or semi-limp and may thus be folded or otherwise kept in a stored state. The limp state of the needle 126 prior to inflation facilitates avoidance of inadvertent body penetration. To ready the needle 126 for use, pressurized fluid (e.g., air) is provided from the pressure source or sources 346 into the channel 344a in the needle wall 344. As the pressure builds, the needle 126 inflates and becomes rigid. Reinforcement members 352 may be provided to enhance rigidity upon inflation. Once inflated, the needle 126 is of sufficient rigidity to permit body penetration. Optionally, the needle 126 may be inflated to a first state for penetration. Then, after penetration, the needle 126 may be further inflated to a second state. The further inflation expands the size of the needle, which may facilitate sealing the point of body penetration. In a further example, the inflatable needle 126 may be utilized as a cover over a traditional needle. For instance, after the traditional needle penetrates the body, the needle 126 is inflated around the traditional needle for sealing at the penetration site. In a further example, the inflatable needle 126 may be inflated to facilitate improved electrical contact with surrounding tissue, useful, for example, when the needle is a conduit path for current or taking biophysical, biochemical or physiological measurements.

FIG. 11B illustrates a similar inflatable needle 126 incorporating similar features as FIG. 11A and FIG. 11C with a localized expansion and contraction region 354 useful for blocking reverse fluid flow past the needle body 344 during an injection event.

FIG. 11C illustrates an inflatable needle 126 incorporating similar features as FIG. 11A and 11B with an additional localized expansion and contraction area 356 of the sharp surface useful for preventing sharps injuries during the injection process.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

1. A device comprising:
a pump body having at least one variable-size compartment, the pump body configured to be actuated to change the size of the at least one variable-size compartment;
flexible bladders disposed in the at least one compartment; and
at least one common port fluidly connected with interiors of the flexible bladders, wherein the change in the size of the at least one variable-size compartment either compresses or expands the flexible bladders in unison and thereby, respectively, either expels the interiors through the at least one common port or draws a vacuum into the interiors through the at least one common port.

2. The device as recited in claim 1, wherein the at least one variable-size compartment includes a plurality of variable-size compartments, the pump body includes a first rack having first arms and a second rack having second arms that are interleaved with the first arms so as to provide the variable-size compartments between the first arms and the second arms, and the flexible bladders are disposed, respectively, in the variable-size compartments.

3. The device as recited in claim 2, wherein actuation of the pump body moves the first arms toward the second arms and thereby reduces the sizes of the variable-size compartments to compress the flexible bladders.

4. (canceled)

5. (canceled)

6. (canceled)

7. The device as recited in claim 1, further comprising at least one needle connected with the at least one common port.

8. The device as recited in claim 1, wherein actuation of the pump body to reduce the size of the at least one variable-size compartment compresses the flexible bladders, and the flexible bladders contain a liquid medicament that is expelled through the common port.

9. (canceled)

10. The device as recited in claim 1, wherein the interiors of the flexible bladders are fluidly connected in series with each other.

11. (canceled)

12. The device as recited in claim 1, wherein the interiors of the flexible bladders are fluidly connected in parallel with the common port.

13. The device as recited in claim 1, further comprising an actuator operable to actuate the pump body.

14. (canceled)

15. (canceled)

16. The device as recited in claim 1, wherein at least two of the flexible bladders are of unequal volume.

17. The device as recited in claim 1, wherein the flexible bladders are formed of a multi-layer bladder wall.

18. The device as recited in claim 1, further comprising an inflatable needle fluidly connected with the at least one port.

19. The device as recited in claim 1, wherein one of the flexible bladders contains a first medicament and the another one of the flexible bladders contains a second, different medicament.

20. The device as recited in claim 1, further comprising a needle fluidly connected with the at least one port and a pad that is adhesively attachable to tissue, the pad having first and second electrodes that are spaced apart from each other, an electric power source connected with the first and second electrodes, and a through hole for receiving the needle there through into the tissue.

21. The device as recited in claim 1, further comprising a needle connected with the at least one port, the needle including an electrical circuit that is open by default, and an electric power source connected with the needle, wherein insertion of the needle into a location of a subject animal to inject a medicament through the needle closes the circuit and produces an electric field in a vicinity of the location that dilates blood vessels and stimulates muscle contractions to aid in delivery of the medicament to the subject animal.

22. The device as recited in claim 1, further comprising a plenum fluidly interconnecting the flexible bladders to the at least one common port.

23. A device comprising:
an actuator;
a pump comprising a pump body including a first rack having first arms and a second rack having second arms that are interleaved with the first arms such that there are compartments between the first arms and the second arms, the first rack and the second rack being moveable relative to each other such that compartments are variable in size;
flexible bladders disposed in the compartments, the flexible bladders containing at least one liquid;
a plenum fluidly interconnecting interiors of the flexible bladders; and
at least one common port fluidly connected with the interiors of the flexible bladders via the plenum,
wherein the actuator is operable to actuate the pump body and thereby cause the first arms to move closer to the second arms to reduce the size of the compartments and thus compress the flexible bladders to expel the liquid from the interiors through the plenum to the at least one common port.

24. The device as recited in claim 23, wherein the actuator comprises a bias member that has stored potential energy.

25. The device as recited in claim 24, wherein the bias member includes a spring.

26. The device as recited in claim 24, wherein the flexible bladders include in the interiors a shape recovery member configured to expand the flexible bladders after compression.

27. A method for using a device, the method comprising:
providing a device that includes:
a pump body having at least one variable-size compartment, the pump body configured to be actuated to change the size of the at least one variable-size compartment,
flexible bladders disposed in the at least one compartment, and
at least one common port fluidly connected with interiors of the flexible bladders;
actuating the pump body to thereby change the size of the at least one variable-size compartment, wherein actuation of the pump body compresses or expands the flexible bladders in unison and thereby, respectively, either expels the interiors through the at least one common port or draws a vacuum into the interiors through the at least one common port.
